(19) United States
(12) Patent Application Publication
(10) Pub. No.: US 2002/0004500 A1
(43) Pub. Date: Jan. 10, 2002

(54) Vasopeptidase Inhibitors to treat isolated systolic hypertension

(76) Inventors: Richard A. Reeves, Pennington, NJ (US); Robert A. Wolf, Newton, PA (US); Paul I. Chang, Doylestown, PA (US)

Correspondence Address: MARLA J MATHIAS, BRISTOL-MYERS SQUIBB COMPANY, PATENT DEPARTMENT, P O BOX 4000, PRINCETON, NJ, 08543-4000, US

(21) Appl. No.: 09/819,549

(22) Filed: Mar. 28, 2001

Related U.S. Application Data
(60) Provisional application No. 60/194,499, filed on Apr. 3, 2000.

(51) Int. Cl. A61K 31/55

(52) U.S. Cl. 514/212.03

(57) ABSTRACT

Vasopeptidase inhibitors, especially omapatrilat, are useful in treating isolated systolic hypertension. The vasopeptidase inhibitor may be used in combination with other pharmaceutically active agents.

Priority is claimed from U.S. provisional application 60/194,499 filed Apr. 3, 2000.

### BACKGROUND OF THE INVENTION

Over the last several years compounds have been reported in the patent and technical literature as possessing in a single molecule both angiotensin converting enzyme (ACE) inhibitory activity and neutral endopeptidase (EC24.11; NEP) inhibition activity. These compounds are of interest as cardiovascular agents particularly in the treatment of hypertension, congestive heart failure, and renal disease. These compounds are also referred to as vasopeptidase, dual metalloprotease, NEP/ACE, or ACE/NEP inhibitors.

Omapatrilat is such a vasopeptidase inhibitor which is currently undergoing clinical evaluation. Omapatrilat has the chemical name [4S-[4α(R*), 7α, 10aβ]]-octahydro-4-[(2-mercapto-1-oxo-3-phenylpropyl)amino]-5-oxo-7H-pyrido[2,1-b][1,3]thiazepine-7-carboxylic acid and the structural formula

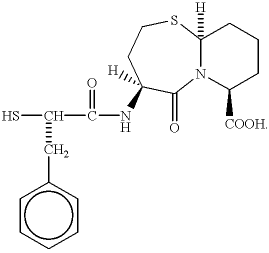

Omapatrilat, its preparation, and its use in treating cardiovascular diseases are disclosed by Robl in U.S. Pat. No. 5,508,272.

BMS 189,921 (gemopatrilat) is another vasopeptidase inhibitor which is currently undergoing clinical evaluation. BMS 189,921 has the chemical name [S-(R*,R*)]-hexahydro-6-[(2-mercapto-1-oxo-3-phenylpropyl)amino]-2,2-dimethyl-7-oxo-1H-azepine-1-acetic acid and the structural formula

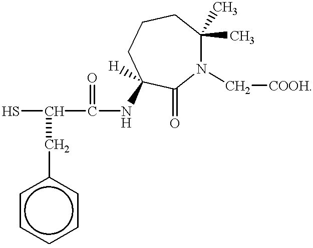

BMS 189,921, it preparation, and its use in treating cardiovascular diseases are disclosed by Karanewsky et al. in U.S. Pat. No. 5,552,397.

### SUMMARY OF THE INVENTION

This invention is directed to the use of a vasopeptidase inhibitor to treat isolated systolic hypertension. Preferred vasopeptidase inhibitors for this use are omapatrilat or a pharmaceutically acceptable salt thereof, BMS 189,921 or a pharmaceutically acceptable salt thereof, or mixtures thereof. Most preferred is the use of omapatrilat.

The vasopeptidase inhibitor or inhibitors can also be employed in combination with other types of pharmaceutically active agents such as other types of antihypertensive agents and/or agents known to be useful in reducing the frequency or severity of stroke and/or coronary disease. The combination therapy can utilize a single dose form containing the vasopeptidase inhibitor or inhibitors or a pharmaceutically acceptable salt thereof, and the other pharmaceutically active agent or agents, co-administration of separate doses of each active agent, or administration of separate doses of each active agent according to a staggered schedule.

### DETAILED DESCRIPTION OF THE INVENTION

There is compelling evidence that systolic blood pressure is at least as important a determinant of risk for stroke and/or coronary disease as is diastolic blood pressure. While the risk for such diseases are continuously related to blood pressure, it is useful to classify hypertension into stages of severity. A widely used classification scheme appears below:

| | Systolic Blood Pressure | Diastolic Blood Pressure |
|---|---|---|
| | Criteria (mm Hg) | Criteria (mm Hg) |
| Stage 1 | 140-159 | 90-99 |
| Stage 2 | 160-179 | 100-109 |
| Stage 3 | ≧180 | ≧110 |

Stage 1 isolated systolic hypertension (systolic blood pressure 140-159 mm Hg with diastolic blood pressure less than 90) is the most common type of untreated hypertension. The prevalence of Stage 1 isolated systolic hypertension among adults over 60 years of age is in the range of 15 to 20%. Of all hypertensive patients over the age of 60 years (regardless of stage), 45-50% have stage 1 isolated systolic hypertension. Elderly, untreated patients with Stage 1 isolated systolic hypertension who are free of clinically apparent cardiovascular disease have a higher prevalence of subclinical cardiovascular disease such as silent myocardial ischemia, increased left ventricular mass, and abnormal diastolic function. Patients with Stage 1 isolated systolic hypertension are at increased risk for subsequent cardiovascular morbidity and mortality by 2- to 4-fold for men and women, respectively. The risk factor for cardiovascular disease is even greater in diabetic patients with isolated systolic hypertension.

This invention is directed to the use of one or more vasopeptidase inhibitors to treat patients with isolated systolic hypertension and thereby reduce cardiovascular mobidity and mortality including fatal and non-fatal stroke, fatal and non-fatal myocardial infarction, fatal and non-fatal heart failure, and other atherosclerotic events. In addition, the early and effective treatment of isolated systolic hypertension may prevent the onset of atrial fibrillation, may prevent the onset of renal failure particularly in diabetic patients, and may slow or halt the progression in the stage of hypertension.

Various angiotensin converting enzyme inhibitors have been reported in the literature as being useful in the treatment of isolated systolic hypertension including captopril, fosinopril, enalapril, lisinopril, lisinopril plus hydrochlorothiazide, ramipril, quinapril and spirapril. The use of vasopeptidase inhibitors according to this invention results in an increased lowering of systolic blood pressure as compared to the above agents which only inhibit the angiotensin converting enzyme. Of added importance, the vasopeptidase inhibitors to a greater extent then angiotension converting enzyme inhibitors are effective in lowering systolic blood pressure in both Caucasian and Black patients. Angiotensin converting enzyme inhibitors including those listed above are generally less effective in lowering systolic and diastolic blood pressure in Black patients.

Preferred vasopeptidase inhibitors for use in treating isolated systolic hypertension according to this invention are omapatrilat or a pharmaceutically acceptable salt thereof, and BMS 189,921 or a pharmaceutically acceptable salt thereof, particularly omapatrilat. The vasopeptidase inhibitor can be administered to a patient suffering from isolated systolic hypertension in an amount ranging from about 2.5 mg to about 240 mg per 24 hours, preferably from about 20 to about 100 mg per 24 hours. The vasopeptidase inhibitor can be administered in one or more doses over the 24 hour period to provide the total amount of active agent within the above range. If more than one dose is administered per 24 hours, the doses may be equal or may be varied in strength. Of course, the amount of active agent employed will be adjusted by the physician according to the severity of the isolated systolic hypertension and its response to the treatment. Also, if a combination of vasopeptidase inhibitors is employed, then one or both of the inhibitors may be administered in a lesser amount provided that the total combination of active agents administered is within the above range.

The vasopeptidase inhibitor is preferably administered orally in tablet or capsule form. However, other methods of administration may also be utilized including sublingually, bucally, parenterally such as by subcutaneous, intraveneous, or intramuscular injection or infusion techniques, nasally such as by inhalation spray, topically such as in the form of a cream or ointment, transdermally as in the form of a patch that is applied to the skin, or rectally such as in the form of suppositories. The various dosage formulations contain in addition to the vasopeptidase inhibitor conventional pharmaceutically acceptable vehicles, stabilizers, preservatives, lubricants, diluents, and other conventional ingredients. The formulation may be administered for immediate release or extended release.

Another aspect of this invention is the treatment of isolated systolic hypertension with one or more vasopeptidase inhibitors, as described above, in combination with other types of pharmaceutically active agents. For example, other antihypertensive agents can be utilized in combination with the vasopeptidase inhibitors. Suitable agents include diuretics such as hydrochlorothiazide, which is preferred, and bendroflumethiazide, α- and/or β-adrenergic blocking agents such as propranolol hydrochloride, timolol maleate, carvedilol, metoprolol tartrate and atenolol, calcium entry blockers such as amlodipine besylate, diltiazem hydrochloride, and verapamil hydrochloride, and angiotensin II receptor antagonists such as irbesartan, losartan, valsartan, candesartan cilexetil, and eprosartan. Agents known to be useful in reducing the frequency or severity of stoke and/or coronary disease can also be utilized in combination with the vasopeptidase inhibitors. Suitable agents include cholesterol reducing agents particularly HMG-CoA reductase inhibitors such as pravastatin sodium, simvastatin, lovastatin, atorvastatin calcium, cerivastatin sodium, and fluvastatin sodium and platelet aggregation inhibitors such as clopidogrel bisulfate, ticlopidine hydrochloride and aspirin.

In such combination therapies, the amount of other pharmaceutically active agents employed is that previously approved for the treatment of hypertension or the reduction of the frequency or severity of stroke and/or coronary disease. Lesser amounts of the other pharmaceutically active agent may be employed as determined by the treating physician. Also, in the combination therapy, the amount of vasopeptidase inhibitor may be less than the amount employed in the monotherapy described above.

The vasopeptidase inhibitor and the other pharmaceutically active agent or agents may be formulated as a single dosage form, may be co-administered from separate dosage forms, or may be administered from separate dosage forms according to a staggered schedule.

The term pharmaceutically acceptable salt includes alkali metal salts such as sodium and potassium, alkaline earth metal salts such as calcium and magnesium, salts derived from amino acids such as arginine, lysine, etc. and salts derived from amines such as alkylamines, e.g. t-butylamine, t-amylamine, etc., substituted alkylamines, e.g. benzylamine, dialkylamines, substituted dialkylamines, e.g. N-methyl glucamine, trialkylamines, substituted trialkylamines, and quaternary ammonium salts.

### EXAMPLE

Omapatrilat was studied in subjects with isolated systolic hypertension (seated systolic blood pressure 160-199 mm Hg and diastolic blood pressure less than 90 after 4 weeks of single-blind placebo lead-in). Both a forced-dose titration (at Week 1) and an elective-dose titration (at Week 9) design were used to compare the change from baseline, relative to placebo, in trough (24±3 hours post dose) seated systolic blood pressure after 9 weeks and 13 weeks of once-daily oral administration of 3 dose regimens of omapatrilat. Treatment regimens consisted of omapatrilat 10 mg lead-in maintained at 10 mg at week 1, electively titrated to 20 mg at week 9 for subjects with a seated systolic blood pressure greater than or equal to 140 mm Hg, or omapatrilat 20 mg lead-in maintained at 20 mg at week 1, electively titrated to 40 mg at week 9 for subjects with a seated systolic blood pressure greater than or equal to 140 mm Hg, or omapatrilat 20 mg lead-in increased to 40 mg at week 1, electively titrated to 80 mg at week 9 for subjects with a seated systolic blood pressure greater than or equal to 140 mm Hg. Placebo was mock titrated at weeks 1 and week 9.

429 subjects were randomized. The mean age was 67 years, and mean baseline blood pressure was 169/82 mm Hg. The primary efficacy objective was to compare the change from baseline, relative to placebo, in trough (24±3 hours post dose) seated systolic blood pressure after 9 weeks of once-daily oral administration of 3 dose regimens of omapatrilat.

At week 9, all 3 omapatrilat regimens produced greater systolic and diastolic blood pressure reductions than the placebo regimen.

| | | Omapatrilat | Omapatrilat | Omapatrilat |
|---|---|---|---|---|
| | Placebo | 10/10 mg | 20/20 mg | 20/40 mg |
| N | 99 | 95 | 89 | 82 |
| Δtrough | −14.2 | −15.9 | −23.1 | −26.1 |
| seated | | | | |
| systolic | | | | |
| blood | | | | |
| pressure | | | | |
| Δtrough | −0.5 | −1.8 | −2.6 | −4.6 |
| seated | | | | |
| diastolic | | | | |
| blood | | | | |
| pressure | | | | |

Additional small reductions in systolic blood pressure versus placebo were seen at Week 13.

Among those subjects with a blood pressure measurement at week 9, the percentage of normalized subjects (trough seated systolic blood pressure less than 140 mm Hg) for omapatrilat 10/10 mg was 28%; for omapatrilat 20/20 mg was 51%; and for omapatrilat 20/40 mg was 47%. Once daily omapatrilat at 80 mg produced persistent elevation in urinary atrial natriuretic peptide excretion rate at 24 hours after dosing consistent with long acting inhibition of neutral endopeptidase.

A forced-titration and elective-titration regimen of omapatrilat 10/10/20 mg, 20/20/40 mg and 20/40/80 mg given once-daily for 13 weeks to predominately elderly subjects with isolated systolic hypertension produced greater systolic blood pressure and diastolic blood pressure reductions at trough than a regimen of placebo.

What is claimed is:

1. A method of treating isolated systolic hypertension comprising administering an effective amount of a vasopeptidase inhibitor.

2. A method of claim 1 wherein said vasopeptidase inhibitor is selected from the group consisting of omapatrilat or a pharmaceutically acceptable salt thereof, [S-(R*,R*)]-hexahydro-6-[(2-mercapto-1-oxo-3-phenylpropyl)amino]-2,2-dimethyl-7-oxo-1H-azepine-1-acetic acid or a pharmaceutically acceptable salt thereof, and mixtures thereof.

3. The method of claim 2 wherein said vasopeptidase inhibitor is omapatrilat.

4. A method of treating isolated systolic hypertension comprising administering an effective amount of a vasopeptidase inhibitor together with another pharmaceutically active agent.

5. A method of claim 4 wherein said vasopeptidase inhibitor is selected from the group consisting of omapatrilat or a pharmaceutically acceptable salt thereof, [S-(R*,R*)]-hexahydro-6-[(2-mercapto-1-oxo-3-phenylpropyl)amino]-2,2-dimethyl-7-oxo-1H-azepine-1-acetic acid or a pharmaceutically acceptable salt thereof, and mixtures thereof.

6. The method of claim 5 wherein said vasopeptidase inhibitor is omapatrilat.

7. A method of claim 4 wherein said other pharmaceutically active agent is co-administered with said vasopeptidase inhibitor.

8. A method of claim 4 wherein said other pharmaceutically active agent is administered separately from said vasopeptidase inhibitor.

9. A method of claim 4 wherein said other pharmaceutically active agent is an antihypertensive agent and/or an agent known to be useful in reducing the frequency or severity of stroke and/or coronary disease.

10. A method of claim 9 wherein said antihypertensive agent is selected from the group consisting of diuretics, α- and/or β-adrenergic blocking agents, calcium entry blockers, angiotensin II receptor antagonists, and said agent known to be useful in reducing the frequency or severity of stroke and/or coronary disease is selected from the group consisting of HMG-CoA reductase inhibitors and platelet aggregation inhibitors.

11. A method of claim 10 wherein said diuretic is hydrochlorothiazide, said α- or β-adrenergic agent is selected from the group consisting of propanolol hydrochloride, timolol maleate, metoprolol tartrate, carvedilol, and atenolol, said calcium entry blocker is selected from the group consisting of amlodipine besylate, diltiazem hydrochloride, and verapamil hydrochloride, said angiotensin II receptor antagonist is selected from the group consisting of irbesartan, losartan, valsartan, candesartan cilexetil, and eprosartan.

12. A method of claim 10 wherein said HMG-CoA reductase inhibitor is selected from the group consisting of pravastatin sodium, simvastatin, lovastatin, atorvastatin calcium, cerivastatin sodium and fluvastatin sodium, and said platelet aggregation inhibitor is selected from the group consisting of clopidogrel bisulfate, ticlopidine hydrochloride and aspirin.

13. A method of claim 6 where said other pharmaceutically active agent is hydrochlorothiazide.
